(19) United States
(12) Patent Application Publication
(10) Pub. No.: US 2005/0064027 A1
(43) Pub. Date: Mar. 24, 2005

(54) Bioadhesive drug delivery system with enhanced gastric retention

(75) Inventors: Jules S. Jacob, Taunton, MA (US); Edith Mathiowitz, Brookline, MA (US); David J. Enscore, Sudbury, MA (US); Marcus A. Schestopol, Providence, RI (US)

Correspondence Address: PATREA L. PABST;PABST PATENT GROUP LLP, 400 COLONY SQUARE, SUITE 1200, ATLANTA, GA, 30361, US

(73) Assignee: Spherics, Inc.

(21) Appl. No.: 10/498,661

(22) PCT Filed: Dec. 13, 2002
(86) PCT No.: PCT/US02/40025

Related U.S. Application Data
(60) Provisional application No. 60/340,402, filed on Dec. 15, 2001.

(51) Int. Cl. A61K 31/522; A61K 9/48; A61K 9/20; A61K 9/16; A61K 9/50

(52) U.S. Cl. 424/451; 424/490; 514/263.38; 424/464

(57) ABSTRACT

Bioadhesive macrosphere delivery systems (“BDDS”) having prolonged gastric retention time due to bioadhesion rather than physical density or size are described. In general, the macrospheres have diameters that are greater than 200 microns, more preferably greater than 500 microns. The bioadhesive macrospheres are released in the stomach where they reside in close proximity to the gastric mucosa for a prolonged period of time. Increased residence of BDDS in the upper GI can lead to increased systemic absorption of drug in the preferred site of systemic absorption, namely the upper GI tract (upper to mid-jejunum). The BDDS may be engineered either as a capsule with drug delivery controlled by a diffusion-limited membrane or degradable shell, or as a solid matrix system with drug delivery controlled by a combination of diffusion and polymer degradation kinetics.

### BACKGROUND OF THE INVENTION

The invention is in field of controlled delivery of therapeutic agents and more specifically concerns the delivery of drugs by the oral route of administration.

It is generally accepted that the oral route of administration is preferred over parenteral administration by patients and has the highest degree of patient compliance. The use of bioadhesive drug delivery systems (BDDS) offers important advantages for oral dosing. Bioadhesive systems can be engineered to have increased residence time in the intestinal tract, which translates into increased local concentrations of therapeutic agents at the residence sites. For purposes of local or topical drug delivery, the increased residence time of BDDS often reduces the frequency of dosing, resulting in improved patient compliance, or else reduces the amount of drug required for dosing, resulting in a reduction of drug-related side-effects.

An additional benefit of BDDS is derived from the close apposition of the BDDS to the target mucosa. The intimate contact of dosage form with mucosa reduces the distance required for drug uptake or drug action. The drug is delivered to the target tissue in a controlled manner and not diluted or deactivated by the contents of the gut lumen. This feature is especially important when the drug is a sensitive protein or DNA-based drug that can be readily deactivated by the harsh conditions of the intestinal tract. Proteins can be denatured by acid gastric pH or else hydrolyzed by a variety of proteases secreted by the gastric mucosa and pancreas.

However for drugs that are not susceptible to proteolysis, degradation or deactivation, such as small organic molecules (SOM), adhesion of drug-loaded BDDS to the stomach and upper GI offers many advantages over conventional DDS. The increased residence time of BDDS in stomach and upper intestinal segments may serve as a platform for delivery of SOM to lower intestinal segments. Drug that is not delivered topically to the site of BDDS residence can flow downstream and be absorbed by jejunal mucosa. Since the upper GI is the primary site for most oral drug absorption and systemic delivery, the benefits of controlled release of drug and bioadhesion result in maintenance of serum drug levels within the therapeutic “window” for longer periods of time than simple bolus dosing.

BDDS are controlled delivery systems where the therapeutic agent is encapsulated either as: (1) a matrix-type device consisting of drug encapsulated in polymer with bioadhesive properties or else containing excipients that increase bioadhesive properties of the system or (2) a diffusion-controlled system comprising a core of drug surrounded by a rate-limiting membrane. The membrane may contain bioadhesive polymers or excipients to increase adhesion to target mucosa.

The scientific and patent literature details a variety of drug delivery systems demonstrating increased gastric retention based not upon bioadhesive properties of the delivery system but relying more upon structural-, density- or size-related properties of the drug delivery system. Floating dosage forms with increased gastric residence time were first described by Sheth and Tossounian in U.S. Pat. No. 4,167,558 and consisted of drugs encapsulated in hydrocolloids such as cellulose ethers, notably hydroxypropylethylcellulose. Hydration of the “Hydrodynamically Balanced System” or HBS in the gastric milieu created a gelled hydration boundary layer responsible for the system's flotation characteristics. Encapsulated drug was released by diffusion into the gastric contents after swelling. Gerogianis et al. (Drug Dev. Ind. Pharm., 19:1061-1081 (1993)) demonstrated that floatation properties were linked to increased molecular weight and viscosity of polymers and reduced hydration of the polymers owing to chemical substitutions on the polymer sidechains. Sangekar et al. (Intl. J. Pharm., 35:187-91 (1987)) compared an HBS formulation to a non-floating formulation and demonstrated that gastric emptying of the dosage forms were related to food and not the floating-properties of the dosage forms. Commercially available HBS formulations include Madopar CR (Roche) for delivery of L-dopa and benserazide and Valrease (Roche) for delivery of diazepam. Both formulations provided for more consistent systemic levels of drug and resulted in reduced dosing in human volunteers (Fell et al., Pharm. Tech. 24:82-91 (2000)).

Gas-generating dosage forms have been used to provide flotation properties. The gas generated is typically carbon dioxide derived from exposure of encapsulated, solid bicarbonate to gastric acidity, and is entrapped in a gel matrix. Yang and Fassihi (J. Pharm. Sci. 85: 170-73 (1996)) described a three-layer, gas-generating tablet that demonstrated buoyancy for up to 16 hrs. in simulated gastric fluid (SGF) in vitro. Agyilirah et al. (Int. J. Pharm. 75:241-7 (1991) described a coated tablet formulation whose coating detached and swelled to more than six-times the original size upon exposure to SGP.

Fell et al., ibid., describe a floating alginate bead system produced by freeze-drying. Alginate beads were produced by ionic gelatin in a calcium chloride bath, frozen and lyophilized. The resulting beads were porous and floated compared to beads dried in an oven. When tested in human volunteers, the solid beads resided in the stomach for 1 hr. while the hollow beads emptied after three hours. The prior art in the field for floating or hollow dosage forms is extensive. However, the degree of bioadhesiveness for these dosage forms is a function of size, density, and/or structure. Therefore the size of and materials for the particles are limited.

It is therefore an object of the present invention to provide improved bioadhesive formulations for oral administration.

It is another object of the invention to provide improved macrosphere formulations that can be encapsulated in capsules, wherein the macrospheres can have different release properties or contain different bioactive compounds.

### SUMMARY OF THE INVENTION

Bioadhesive macrosphere delivery systems (“BDDS”) have been developed having prolonged gastric retention time due to bioadhesion rather than physical density or size. In general, the macrospheres have diameters that are greater than 200 microns, more preferably greater than 500 microns. The bioadhesive macrospheres are released in the stomach where they reside in close proximity to the gastric mucosa and do not float in the gastric contents. The mechanism of increased gastric retention is due to increased adhesion of the delivery system to gastric mucosa in the stomach and upper small intestine, where they reside for an extended period of time, as demonstrated by the examples, and are capable of delivering drugs locally or topically in the gastric compartment. As a result of the increased residence of BDDS in the upper GI, drug not absorbed at the site of residence can be directed to lower GI segments over long periods of time. The directed “overflow” of drug from a resident BDDS can lead to increased systemic absorption of drug in the preferred site of systemic absorption, namely the upper GI tract (upper to mid-jejunum).

The BDDS may be engineered either as a capsule with drug delivery controlled by a diffusion-limited membrane or degradable shell, or as a solid matrix system with drug delivery controlled by a combination of diffusion and polymer degradation kinetics. One embodiment comprises a capsule or microcapsule ranging in size from 0.1 to 2.5 mm in diameter consisting of a solid core of drug, hydrophilic polymer binder and excipients coated with a rate-limiting membrane and a bioadhesive membrane. In one preferred embodiment, the core consists of drug in concentrations of 40-95% w/w. The cores may be manufactured using any of a number of techniques including but not limited to ionic gelation, hot-melt, melt-granulation, extrusion-spheronization, wet granulation, fluid-bed agglomeration etc. Alternatively, the cores may consist of commercially available “non-pareils”, e.g. SugarSphere, USP, to which the drug and polymer coating may be applied using different coating processes.

### BRIEF DESCRIPTION OF THE DRAWINGS

FIG. 1 is a flow chart of the production of macrosphere drug delivery devices, beginning with wet mixing, extrusion, spheronization, drying, and coating.

FIG. 2 is a graph of enhanced GI residence time of macrospheres versus release time (hours). Four preparations were compared: A, the control macrospheres; B, macrospheres with a coating of fumaric acid pre-polymer (“FAPP”), with molecular weight less than 500 Da and Fe₃O₄; C, macrospheres with a coating of fumaric acid-sebacic acid copolymer (“FA:SA”) 20:80, with a molecular weight less than 20,000 Da, and FAPP; D, macrospheres with a coating of FA:SA, FAPP and CaO.

FIG. 3 is a graph of acyclovir concentration in serum (μg/ml) versus time (hours) after dosing in dogs. Formulation #1 (designated by ⋄) contained 5% of the total acyclovir loading incorporated in the bioadhesive coating, while Formulation #2 (designated by ▴) contained acyclovir only in the core.

FIG. 4 is a bar graph comparing area under the curve values (μg/ml*hr) for Formulations #1 (left bar) and #2 (right bar). These values were calculated from the data in FIG. 3.

FIG. 5 is a bar graph comparing the residence time for the microspheres in Formulations #1 (left column) and #2 (right bar) in the upper GI of the dogs.

FIG. 6 is a graph of release of acyclovir (ACV) and salicylate as a function of percent total acyclovir loading, over time (in hours), from macrospheres wherein the salicylate is encapsulated in an outer Eudragit® RL 100 coating and the acyclovir is encapsulated in the core. The outer drug loading is used to achieve rapid release (three hours) as compared to more long term release of the core drug (24 hours).

### DETAILED DESCRIPTION OF THE INVENTION

The BDDS described herein consists of macrospheres, which include at least a therapeutic, diagnostic or prophylactic agent to be delivered, bioadhesive elements (which may be polymers, metal oxides, or ligands for specific mucosal components), and release controlling materials, which may effect release by degradation, diffusion, pH, or a combination thereof

The macrospheres are typically in the range of from 0.1 to 3 mm in diameter, preferably greater than 0.2 mm, most preferably greater than 0.5 mm. They typically contain one or more agents to be delivered and one or more rate controlling materials, for example, rate controlling membranes. In some embodiments there are multiple therapeutic agents released at different times. In other embodiments, therapeutic agent is released from the rate controlling membrane as well as from the core of the macrosphere, where the therapeutic agent in the membrane may be the same or different from the agent in the core. macrospheres can be administered as a powder, encapsulated within a gelatin or enteric coating, or compressed into a tablet. macrospheres of the same or different carrier composition or active agent can be mixed together in a single formulation.

The macrospheres can contain between 10 and 70% of therapeutic, diagnostic or prophylactic agent (referred to hereafter as “active”) by weight of macrosphere, or between 30 and 90% by weight of the core of a coated macrosphere, where each coating makes up between 1-10% , preferably 5-6%, by weight of the macrosphere, up to a total of about 30% by weight of the macrosphere. The coating can include active, in ratios of between 5 and 50% by weight of the coating, preferably between 20 and 40% by weight of the coating, while still retaining rate control.

Polymers Useful in Forming Bioadhesive Particles

Suitable polymers that can be used to form bioadhesive particles include soluble and insoluble, biodegradable and nonbiodegradable polymers. These can be hydrogels or thermoplastics, homopolymers, copolymers or blends, natural or synthetic. The preferred polymers are synthetic polymers, with controlled synthesis and degradation characteristics. Most preferred polymers are copolymers of fumaric acid and sebacic acid, which have unusually good bioadhesive properties when administered to the gastrointestinal tract. Other preferred polymers suitable for use in these systems include degradable polymers: polyesters such as poly-lactic acid (PLA), poly(lactide-co-glycolide) or PLGA, polycaprylactone (PCL); polyanhydrides such as poly(fumaric-co-sebacic) in molar ratios of 20:80 to 90:10, poly(carboxyphenoxypropane-co-sebacic acid (PCPP:SA); polyorthoesters; polyamides; and polyamides. Other suitable polymers include hydrogel based polymers such as agarose, alginate, chitosan etc. and non-degradable polymers such as polystyrene, polyvinylphenol, polymethylmethacrylates (Eudragits®).

Rapidly bioerodible polymers such as poly[lactide-co-glycolide], polyanhydrides, and polyorthoesters, whose carboxylic groups are exposed on the external surface as their smooth surface erodes, are excellent candidates for bioadhesive drug delivery systems. In addition, polymers containing labile bonds, such as polyanhydrides and polyesters, are well known for their hydrolytic reactivity. Their hydrolytic degradation rates can generally be altered by simple changes in the polymer backbone.

Representative natural polymers include proteins, such as zein, modified zein, casein, gelatin, gluten, serum albumin, or collagen, and polysaccharides, such as cellulose, dextrans, polyhyaluronic acid, polymers of acrylic and methacrylic esters and alginic acid. Synthetically modified natural polymers include alkyl celluloses, hydroxyalkyl celluloses, cellulose ethers, cellulose esters, and nitrocelluloses. Representative synthetic polymers include polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Representative bioerodible polymers include polylactides, polyglycolides and copolymers thereof, poly(ethylene terephthalate), poly(butic acid), poly(valeric acid), poly(lactide-co-caprolactone), poly[lactide-co-glycolide], polyanhydrides, polyorthoesters, blends and copolymers thereof.

These polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

Bioadhesive Elements

Polymers can be selected for or chemically modified to increase bioadhesion. For example, the polymers can be modified by increasing the number of carboxylic groups accessible during biodegradation, or on the polymer surface. The polymers can also be modified by binding amino groups to the polymer. The polymers can also be modified using any of a number of different coupling chemistries that covalently attach ligand molecules with bioadhesive properties to the surface-exposed molecules of the polymeric particles.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex. By using either of these protocols it is possible to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Any suitable coupling method known to those skilled in the art for the coupling of ligands and polymers with double bonds, including the use of UV crosslinking, may be used for attachment of bioadhesive ligands to the polymeric particles described herein. Any polymer that can be modified through the attachment of lectins can be used as a bioadhesive polymer for purposes of drug delivery or imaging.

Lectins that can be covalently attached to particles to render them target specific to the mucin and mucosal cell layer could be used as bioadhesives. Useful lectin ligands include lectins isolated from: Abrus precatroius, Agaricus bisporus, Anguilla anguilla, Arachis hypogaea, Pandeiraea simplicifolia, Bauhinia purpurea, Caragan arobrescens, Cicer arietinum, Codium fragile, Datura stramonium, Dolichos biflorus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclurapomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limaxfluvus, Homarus americanus, Cancer antennarius, and Lotus tetragonolobus.

The attachment of any positively charged ligand, such as polyethyleneimine or polylysine, to any particle may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most particles with the appropriate chemistry, such as CDI, and be expected to influence the binding of particles to the gut. For example, polyclonal antibodies raised against components of mucin or else intact mucin, when covalently coupled to particles, would provide for increased bioadhesion. Similarly, antibodies directed against specific cell surface receptors exposed on the lumenal surface of the intestinal tract would increase the residence time of beads, when coupled to particles using the appropriate chemistry. The ligand affinity need not be based only on electrostatic charge, but other useful physical parameters such as solubility in mucin or else specific affinity to carbohydrate groups.

The covalent attachment of any of the natural components of mucin in either pure or partially purified form to the particles would decrease the surface tension of the bead-gut interface and increase the penetration of the bead into the mucin layer. The list of useful ligands would include but not be limited to the following: sialic acid, neuraminic acid, n-acetyl-neurarminic acid, n-glycolylneuraminic acid, 4-acetyl-n-acetylneuraminic acid, diacetyl-n-acetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface.

The attachment of polyamino acids containing extra pendant carboxylic acid side groups, e.g., polyaspartic acid and polyglutamic acid, should also provide a useful means of increasing bioadhesiveness. Using polyamino acids in the 15,000 to 50,000 kDa molecular weight range would yield chains of 120 to 425 amino acid residues attached to the surface of the particles. The polyamino chains would increase bioadhesion by means of chain entanglement in mucin strands as well as by increased carboxylic charge.

The bioadhesive properties of a polymer are enhanced by incorporating a metal compound into the polymer to enhance the ability of the polymer to adhere to a tissue surface such as a mucosal membrane. Metal compounds which enhance the bioadhesive properties of a polymer preferably are water-insoluble metal compounds, such as water-insoluble metal oxides and hydroxides, including oxides of calcium, iron, copper and zinc. The metal compounds can be incorporated within a wide range of hydrophilic and hydrophobic polymers including proteins, polysaccharides and synthetic biocompatible polymers. In one embodiment, metal oxides can be incorporated within polymers used to form or coat drug delivery devices, such as microspheres, which contain a drug or diagnostic agent. The metal compounds can be provided in the form of a fine dispersion of particles on the surface of a polymer that coats or forms the devices, which enhances the ability of the devices to bind to mucosal membranes. As defined herein, a water-insoluble metal compound is defined as a metal compound with little or no solubility in water, for example, less than about 0.9 mg/ml.

The water-insoluble metal compounds, such as metal oxides, can be incorporated by one of the following mechanisms: (a) physical mixtures which result in entrapment of the metal compound; (b) ionic interaction between metal compound and polymer; (c) surface modification of the polymers which would result in exposed metal compound on the surface; and (d) coating techniques such as fluidized bead, pan coating or any similar methods known to those skilled in the art, which produce a metal compound enriched layer on the surface of the device.

Preferred properties defining the metal compound include: (a) substantial insolubility in aqueous environments, such as acidic or basic aqueous environments (such as those present in the gastric lumen); and (b) ionizable surface charge at the pH of the aqueous environment. The water-insoluble metal compounds can be derived from metals including calcium, iron, copper, zinc, cadmium, zirconium and titanium. For example, a variety of water-insoluble metal oxide powders may be used to improve the bioadhesive properties of polymers such as ferric oxide, zinc oxide, titanium oxide, copper oxide, barium hydroxide, stannic oxide, aluminum oxide, nickel oxide, zirconium oxide and cadmium oxide. The incorporation of water-insoluble metal compounds such as ferric oxide, copper oxide and zinc oxide can tremendously improve adhesion of the polymer to tissue surfaces such as mucosal membranes, for example in the gastrointestinal system.

Polymers with enhanced bioadhesive properties can also be obtained by incorporating into the polymer anhydride monomers or oligomers. The polymers may be used to form drug delivery systems which have improved ability to adhere to tissue surfaces, such as mucosal membranes. The anhydride oligomers are formed from organic diacid monomers, preferably the diacids normally found in the Krebs glycolysis cycle. Anhydride oligomers which enhance the bioadhesive properties of a polymer have a molecular weight of about 5000 or less, typically between about 100 and 5000 daltons, or include 20 or fewer diacid units linked by anhydride linkages and terminating in an anhydride linkage with a carboxylic acid monomer.

The oligomer excipients can be blended or incorporated into a wide range of hydrophilic and hydrophobic polymers including proteins, polysaccharides and synthetic biocompatible polymers. In one embodiment, oligomers can be incorporated within polymers used to form or coat drug delivery systems, such as microspheres, which contain a drug or diagnostic agent. In another embodiment, oligomers with suitable molecular weight may be used alone to encapsulate therapeutic or diagnostic agents. In yet another embodiment, anhydride oligomers may be combined with metal oxide particles to improve bioadhesion even more than with the organic additives alone. Organic dyes because of their electronic charge and hydrophobicity/hydrophilicity can either increase or decrease the bioadhesive properties of polymers when incorporated into the polymers.

Formation of Particles

a. Solvent Evaporation. In this method the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid particles. Several different polymer concentrations can be used, including concentrations ranging from 0.05 to 0.20 g/ml. The solution is loaded with a drug and suspended in 200 ml of vigorously stirred distilled water containing 1% (w/v) poly(vinyl alcohol) (Sigma). After 4 hours of stirring, the organic solvent evaporates from the polymer, and the resulting particles are washed with water and dried overnight in a lyophilizer. Particles with different sizes (1-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which are performed in completely anhydrous organic solvents, are more useful.

b. Hot Melt Microencapsulation. In this method, the polymer is first melted and then mixed with the solid particles of dye or drug that have been sieved to less than 50 microns. The mixture is suspended in a non-miscible solvent (like silicon oil), and, with continuous stirring, heated to 5° C. above the melting point of the polymer. Once the emulsion is stabilized, it is cooled until the polymer particles solidify. The resulting particles are washed by decantation with petroleum ether to give a free-flowing powder. Particles with sizes between one to 1000 microns are obtained with this method. The external surfaces of spheres prepared with this technique are usually smooth and dense. This procedure is used to prepare particles made of polyesters and polyanhydrides. However, this method is limited to polymers with molecular weights between 1000-50,000.

c. Solvent Removal. This technique is primarily designed for polyanhydrides. In this method, the drug is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make particles from polymers with high melting points and different molecular weights. Particles that range between 1-300 microns can be obtained by this procedure. The external morphology of spheres produced with this technique is highly dependent on the type of polymer used.

d. Hydrogel Particles. Particles made of gel-type polymers, such as alginate, are produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The particles are left to incubate in the bath for twenty to thirty minutes in order to allow sufficient time for gelation to occur. Particle size is controlled by using various size extruders or varying either the nitrogen gas or polymer solution flow rates.

Chitosan particles can be prepared by dissolving the polymer in acidic solution and crosslinking it with tripolyphosphate. Carboxymethyl cellulose (CMC) particles were prepared by dissolving the polymer in acid solution and precipitating the particle with lead ions. Alginate/polyethylene imide (PEI) were prepared in order to reduce the amount of carboxylic groups on the alginate microcapsule. The advantage of these systems is the ability to further modify their surface properties by the use of different chemistries. In the case of negatively charged polymers (e.g., alginate, CMC), positively charged ligands (e.g., polylysine, polyethyleneimine) of different molecular weights can be ionically attached.

e. Extrusion-Spheronization. Core particles may be prepared by the process of granulation-extrusion-spheronization. In this process, micronized drug is mixed with microcrystalline cellulose, binders, diluents and water and extruded as a wet mass through a screen. The result is rods with diameters equal to the opening of the extrusion screen, typically in the size range of 0.1 to 5 mm. The rods are then cut into segments of approximately equal length with a rotating blade and transferred to a spheronizer. The spheronizer consists of a rapidly rotating, textured plate which propels rod segments against the stationary walls of the apparatus. Over the course of 1-10 minutes of spheronization, the rods are slowly transformed into spherical shapes by abrasion. The resulting spheroid cores are then discharged from the machine and dried at 40-50° C. for 2448 hours using tray-driers or fluidized bed dryers. The cores may then be coated with rate-releasing, enteric or bioadhesive polymers using either pan-coating or fluidized-bed coating devices.

Excipients—Hydrophilic Binders; Diluents

The macrospheres can include other materials, such as hydrophilic binders. Examples include any of the pharmaceutically accepted hydrogels, e.g., alginate, chitosan, methylmethacrylates (e.g. Eudragit®), celluloses (especially microcrystalline cellulose, hydroxypropylmethylcellulose, ethylcellulose etc.), agarose, Providone™. Examples of other excipients include diluents such as lactose, microcrystalline cellulose, kaolin, starch or magnesium stearate, density-controlling agents such as barium sulfate or oils, and rate-controlling agents such as magnesium stearate, oils, ion-exchange resins.

Macrospheres can be incorporated into standard pharmaceutical dosage forms such as gelatin capsules and tablets. Gelatin capsules, available in sizes 000, 00, 0, 1, 2, 3, 4, and 5, from manufactures such as Capsugel®, may be filled with macrospheres and administered orally. Similarly, macrospheres may be dry blended or wet-granulated with diluents such as microcrystalline cellulose, lactose, cabosil and binders such as hydroxypropylmethylcellulose, hydroxypropylcellulose, carboxymethylcellulose and directly compressed to form tablets. The dimensions of the tablets are limited only by the engineering of dies available for tabletting machines. Dies to form tablets in round, oblong, convex, flat, and bullet designs in sizes ranging from 1 to 20 mm are available. The resulting tablets may weigh from 1 to 5,000 mg and carry macrospheres at loadings of 1 to 80% w/w.

The resulting tablets may be coated with sugars, enteric polymers or gelatin to alter dissolution of the tablet and release of the macrospheres into the GI tract. Alternately, tablet diluents may include gas generating elements such as tartaric acid, citric acid and sodium bicarbonate, as examples. Exposure of the tablet to water or gastric fluids facilitates reaction of the weak acid with bicarbonate, resulting in evolution of carbon dioxide. Evolution of gas disrupts the mechanical integrity of the tablet, facilitating release of incorporated macrospheres. Premature dissolution of the tablet in the mouth may be prevented by coating with hydrophilic polymers, such as hydroxypropylmethylcellulose or gelatin, resulting in dissolution in the stomach.

Rate Controlling Elements

Rate control can be achieved by the use of a membrane or diffusion-limiting coating(s), by controlling the rate of degradation of the polymer, and/or the porosity of the macrosphere. Further rate control can be achieved through the use of a capsule such as a gelatin capsule, an enteric coating, and/or tablet size and compression techniques.

The membrane or diffusion-limiting coating can be formed from a variety of different materials including pharmaceutically-accepted polymeric coating materials such as methylmethacrylates (e.g. Eudragit®, Rohm and Haas and Kollicoat®, BASF), zein, cellulose, acetate, cellulose phthalate, hydroxylpropylmethylcellulose, etc. The coatings may be applied using a variety of techniques including fluidized-bed coating, pan-coating and dip-coating. In the preferred embodiment, the coating is applied as a fluidized-bed coating.

Therapeutic, Prophylactic and Diagnostic Agents

Therapeutic agents to be encapsulated include antivirals such as acyclovir and protease inhibitors alone or in combination with nucleosides for treatment of HIV or Hepatitis B or C, anti-parasites (helminths, protozoans), anti-cancer agents (referred to herein as “chemotherapeutic”, including cytotoxic drugs such as cisplatin and carboplatin, BCNU, 5FU, methotrexate, adriamycin, camptothecin, and taxol), antibodies and bioactive fragments thereof (including humanized, single chain, and chimeric antibodies), antigen and vaccine formulations, peptide drugs, anti-inflammatories, oligonucleotide drugs (including antisense, aptamers, ribozymes, external guide sequences for ribonuclease P, and triplex forming agents), antibiotics, antiinflammatories including non-steroidal antiinflammatories (“NSAIDS”) such as methyl salicylate, antiulcerative agents such as bismuth subsalicylate, digestive supplements and cofactors, and vitamins, especially those that are not normally absorbed in the colon. Examples of other useful drugs include ulcer treatments such as Carafate® from Marion Pharmaceuticals, neurotransmitters such as L-DOPA, antihypertensives or saluretics such as Metolazone from Searle Pharmaceuticals, carbonic anhydrase inhibitors such as Acetazolamide from Lederle Pharmaceuticals, insulin like drugs such as glyburide, a blood glucose lowering drug of the sulfonylurea class, synthetic hormones such as Android F from Brown Pharmaceuticals and Testred (methyltestosterone) from ICN Pharmaceuticals, and antiparasitics such as mebendzole (Vermox®, Jannsen Pharmaceutical). Other drugs for application to the vaginal lining or other mucosal membrane lined orifices such as the rectum include spermacides, yeast or trichomonas treatments and anti-hemorrhoidal treatments.

Antigens can be encapsulated in one or more types of bioadhesive polymer to provide a vaccine. The vaccines can be produced to have different retention times in the gastrointestinal tract. The different retention times, among other factors, can stimulate production of more than one type (IgG, IgM, IgA, IgE, etc.) of antibody.

Multiple drug formulations can be prepared either (1) by encapsulating different drugs in coatings/cores or (2) by simply mixing separate batches of particles each containing a single drug to make a new batch containing multiple drugs, as demonstrated by Example 2, in which a model drug, sodium salicylate, is prepared in an outer Eudragit® RL100 coating and a second drug, acyclovir, is prepared in the core. The sodium salicylate is quickly released within 3 hours while the acyclovir has sustained release over the course of 24 hrs.

In a preferred method for imaging, a radio-opaque material such as barium is coated with polymer. Other radioactive materials or magnetic materials could be used in place of, or in addition to, the radio-opaque materials. Examples of other materials include gases or gas-emitting compounds, which are radioopaque.

Barium sulfate suspension is the universal contrast medium used for examination of the upper gastrointestinal tract, as described by D. Sutton, Ed., A Textbook of Radiology and Imaging, Vol. 2, Churchill Livingstone, London (1980), even though it has undesirable properties, such as unpalatability and a tendency to precipitate out of solution. Several properties are critical: (a) Particle size: the rate of sedimentation is proportional to particle size (i.e., the finer the particle, the more stable the suspension); (b) Non-ionic medium: charges on the barium sulfate particles influence the rate of aggregation of the particles, and aggregation is enhanced in the presence of the gastric contents; and (c) Solution pH: suspension stability is best at pH 5.3, however, as the suspension passes through the stomach, it is inevitably acidified and tends to precipitate. The encapsulation of barium sulfate in particles of appropriate size provides a good separation of individual contrast elements and may, if the polymer displays bioadhesive properties, help in coating, preferentially, the gastric mucosa in the presence of excessive gastric fluid. With bioadhesiveness targeted to more distal segments of the gastrointestinal tract, it may also provide a kind of wall imaging not easily obtained otherwise. The double contrast technique, which utilizes both gas and barium sulfate to enhance the imaging process, especially requires a proper coating of the mucosal surface. To achieve a double contrast, air or carbon dioxide must be introduced into the patient's gastrointestinal tract. This is typically achieved via a nasogastric tube to provoke a controlled degree of gastric distension. Studies indicate that comparable results may be obtained by the release of individual gas bubbles in a large number of individual adhesive particles and that this imaging process may apply to intestinal segments beyond the stomach.

Administration of Bioadhesive Particles to Patients

The macrosphere particles are administered to the mucosal membranes, typically via the nose, mouth, rectum, or vagina. In the preferred embodiment, the macrospheres are administered orally. Pharmaceutically acceptable carriers for oral or topical administration are known and can be determined based on compatibility with the polymeric material. Other carriers include bulking agents, such as Metamucil®.

Macrospheres are typically administered in an effective amount based on the agent to be delivered. This amount will be determined based on the known properties and pharmacokinetics of the drugs to be delivered, although this may be adjusted as appropriate in view of the increased residence time, which may enhance the percent uptake of the drug into the gastrointestinal tract.

An in vivo method for evaluating bioadhesion uses encapsulation of a radio-opaque material, such as barium sulfate, or both a radio-opaque material and a gas-evolving agent, such as sodium carbonate, within a bioadhesive polymer. After oral administration of the radio-opaque material, its distribution in the gastric and intestinal areas is examined using image analysis.

The present invention will be further understood by reference to the following non-limiting examples.

### EXAMPLE 1

### Preparation of Macrospheres for Release of Acyclovir

Macrospheres with acyclovir in the cores in an amount of 80% and 90% w/w were made using the wet-granulation/extrusion/spheronization process. The overall yield of the process was 90%, and 90% of the spheronized cores were within the size range of 1.4-2.36 mm.

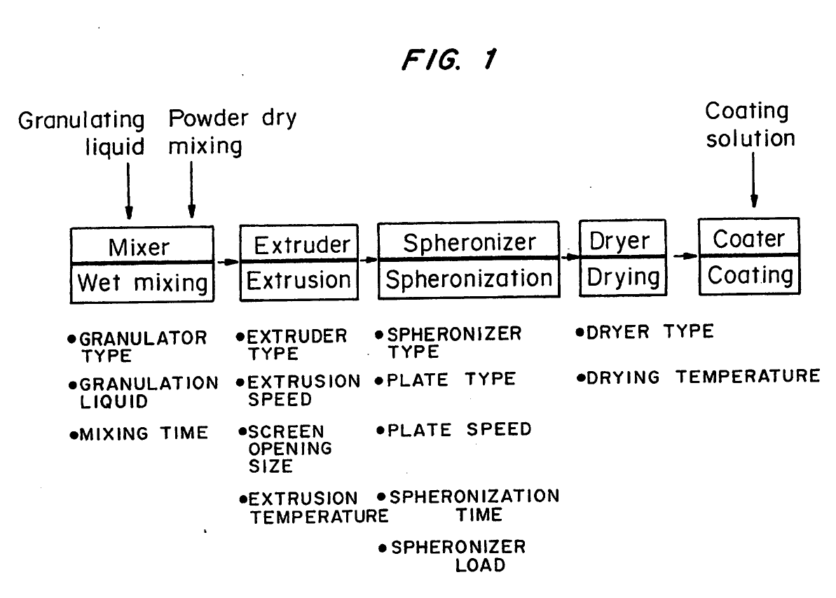

FIG. 1 is a graph of the granulating and spheronization process used to make the macrospheres. Five unit operations are involved in this process. They are (1) wet granulation (making the dough), (2) extrusion of the granulation or “dough” into cylinders, (3) spheronization of the cylinders into spheres, (4) drying, and (5) film coating.

### EXAMPLE 2

### Macrospheres with Modified Release

Release kinetics were obtained from macrospheres with the following compositions: (1) naked drug cores; (2) EUDRAGIT® RL100-coated (diffusion controlling layer) cores and (3) FASA/FAPP/CaO (bioadhesive)-RL100-drug cores. By incorporating drug into the outer bioadhesive coating, nearly first order release kinetics were obtained.

The ability to tailor and optimize drug release is achieved by encapsulating drug in either the bioadhesive (composition #3) or rate-limiting (compositions #2) coating or combinations of the two. It is also possible to spray pure drug onto the surface of the outer coating to achieve a quick burst of available drug. The latter can be demonstrated by spraying RL 100 as a 5% coating over 40% drug-loaded cores. The drug in the coating is sodium salicylate (“Drug 1”); the drug in the core is acyclovir (ACV) (”Drug 2”).

This example demonstrates production of a rate-limiting membrane over the 40% ACV cores. EUDRAGITS® are traditionally used to control release properties of drug-loaded spheres. Spraying RL 100 in the correct concentration gives the desired drug release properties.

Materials/Controls: A 200.4 g lot of beads, 40% w/w Acyclovir (1.4-2.36 mm) was used as the cores for the coatings.

TABLE 1
COMPOSITION OF EUDRAGIT ® RL 100
COATING

| | |
|---|---|
| Liquid | Solid |

| Components | gm | w/w | Gm | w/w |
|---|---|---|---|---|
| Eudragit ® RL 100 | 6 | 5.00% | 6 | 49.59% |
| DBS | 0.6 | 0.50% | 0.6 | 4.96% |
| Talc | 4.9 | 4.08% | 4.9 | 40.50% |
| Mg Stearate | 0.6 | 0.50% | 0.6 | 4.96% |
| DCM | 24 | 19.98% | | |
| IPA | 84 | 69.94% | | |
| Total | 120.1 | 100.00% | 12.1 | 100.00% |

The beads were fluidized at 200 fps with an inlet air temperature of 86° F. using the Wurster setup. The 10″ Wurster tube was used, and set 1″ from the top of the spray nozzle. The coatings were sprayed at an atomization pressure of 10 psi. The formulation exhibited a weight gain of 12.3 g (6.1%). The beads were dried in the fluidized bed for 5 min. The coatings appeared thin and uniform.

Macrospheres containing 30% acyclovir cores were also manufactured. The macrospheres were separated by sieving and the weight of cores (in grams) in a size range was measured. The weight percentage of cores was calculated with respect to the total mass of cores that were sieved. The size ranges (mm), along with their corresponding weight percentages are: greater than 2.36 mm comprised 1% w/w; 1.7-2.36 mm comprised 70% w/w; and less than 1.4 mm comprised 9% w/w. The total recovery of the sieved macrospheres comprised 80% w/w.

### EXAMPLE 3

### Production of Macrospheres with Rate-limiting Membrane and Bioadhesive Coating

Macrospheres containing 30% acyclovir cores were prepared as described in Example 1, with a rate-limiting membrane as described in Example 2, and further coated with a bioadhesive membrane including EUDRAGIT®, calcium oxide, FAPP (anhydride oligomer), and polymer (polyfumaric acid:sebacic acid). The bioadhesive coating is preferably approximately 50 microns in thickness, although coatings can be between 5 and 20 microns, and 5-20% w/w. The bioadhesive coating was applied by fluidized bed coating. Alternatively the coating may be applied by pan coating.

TABLE 2
COMPOSITION OF COATING SOLUTIONS

| | |
|---|---|
| 1^{st} Coat | 2^{nd} Coat |
| Total Solids | Total Solids |

| Component | gm | % w/w | gm | % w/w |
|---|---|---|---|---|
| Eudragit ® RS 100 | 5 | 50 | NA | NA |
| P(FA:SA) | NA | NA | 3 | 15 |
| FAPP | NA | NA | 4 | 24 |
| CaO | NA | NA | 7 | 41 |
| Magnesium Stearate | 1 | 10 | NA | NA |
| Talc | 3.5 | 35 | NA | NA |
| Dibutyl Sebacate | 0.5 | 5 | 1 | 5 |
| Isopropanol | 70 | | 32 | |
| Dichloromethane | 20 | | 50 | |

The function of the materials is as follows: Eudragit® RS 100—Rate-Limiting Polymer; P(FA:SA)—Bioadhesive Polymer, FAPP—Organic Bioadhesive Excipient; CaO—Inorganic Bioadhesive Excipient; Magnesium Stearate—Lubricant; Talc—Glidant; Dibutyl Sebacate—Plasticizer; Isopropanol—Solvent; Dichloromethane—Solvent.

The first coat provided controlled release. The second coat provided a bioadhesive surface.

### EXAMPLE 4

### Retention in Gastrointestinal Tract of Macrospheres

Macrospheres prepared as in Example 3 were administered to dogs and the dogs were x-rayed. The beads contained barium sulfate so that they could be imaged. The cores of the beads were prepared by extrusion/spheronization, with a size range between 1.4 and 2.36 mm, and contained 50% w/w barium sulfate. Control macrospheres were formed with the same composition, but without the bioadhesive coatings. Four preparations were compared: A, the control macrospheres; B, macrospheres with a coating of fumaric acid pre-polymer (“FAPP”), with molecular weight less than 500 Da and Fe₃O₄; C, macrospheres with a coating of fumaric acid-sebacic acid copolymer (“FA:SA”) 20:80, with a molecular weight less than 20,000 Da, and FAPP; D, macrospheres with a coating of FA:SA, FAPP and CaO.

TABLE 3
COMPOSITION OF 30% ACYCLOVIR* (W/W)
MACROSPHERE CORES

| | | % w/w | |
|---|---|---|---|
| Component | Function | Solids | Total |
| Microcrystalline Cellulose | Wet-Massing Excipient | 50 | 35.7 |
| Barium Sulfate | Density/Radiopaque Agent | 17.5 | 12.5 |
| Hydroxypropyl Cellulose | Binder | 2 | 1.4 |
| Acyclovir* | Active | 30 | 21.4 |
| SDS | Extrusion Excipient/ | 0.5 | 0.4 |
| | lubricant | | |
| Water | | | 28.6 |

*Acyclovir was not included in the dog imaging studies, but was added for the release kinetic studies described in Example 5. The weight difference in the dog imaging study was made up by addition of barium sulfate.

3.0 grams of macrospheres dry compressed (2000 psi for 10 seconds in a Stokes DS-3 manual tabletting die) with inert tabletting excipients (1.5 g macrospheres/tablets, 1 gram of lactose, 0.5 g tartaric acid, and 0.5 g sodium bicarbonate) into tablets were administered orally to dogs fasted for 18 hours. Water was given ad libitum. The animals were x-rayed every thirty minutes.

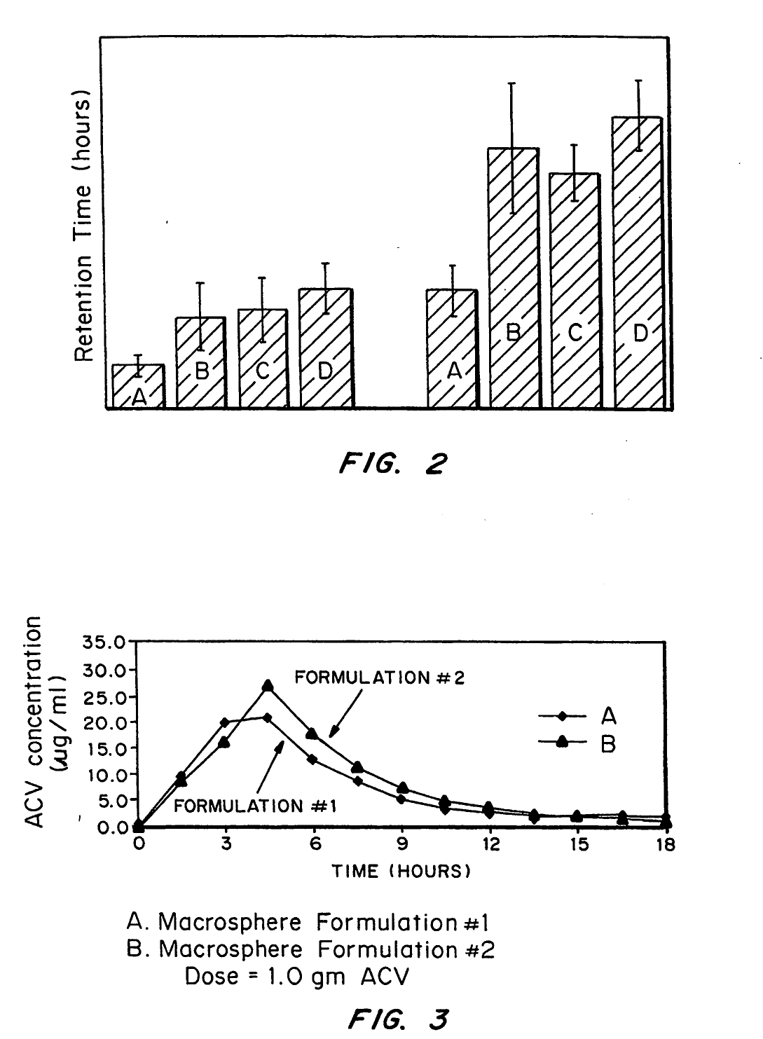

FIG. 2 is a graph comparing the residence times of the bioadhesive macrospheres with the residence times of the control macrospheres. After 30 minutes, the control and bioadhesive macrospheres were just entering the small intestine. After 1.5 hours, the control macrospheres were distributed throughout the small intestine, but the bioadhesive macrospheres were still in the upper portion of the small intestine. After 2.5 hours, the control macrospheres were in the lower portion of the small intestine, while the bioadhesive macrospheres were still in the upper portion of the small intestine. Animals were fed 3.5 hours after dosing. After 6.5 hours, the control macrospheres were passing through the lower portion of the lower intestine, while the bioadhesive macrospheres were just beginning to descend through the small intestine. After 8.5 hours, the bioadhesive macrospheres were distributed throughout the small intestine. After 24 hours, no control macrospheres were detected by x-ray, while the bioadhesive macrospheres were beginning passage through the lower intestine.

### EXAMPLE 5

### In Vitro Release from Macrospheres

The release properties of two macrosphere formulations in simulated gastric fluid at 37° C. are shown in FIG. 3. Formulation #1 had 5% of the total drug loading incorporated in the bioadhesive coating, while Formulation #2 had drug only in the core. The formulations released 40-50% of their load in 6-8 hrs and 100% of the loading in 24 hrs.

### EXAMPLE 6

### In Vivo Release from Macrospheres Tested in Dogs

The formulations in Example 5 were filled into #000 gel caps and orally administered to beagles that had been fasted for 18 hrs. The dose was equivalent to 1.0 gm of acyclovir/dog (˜80-90 mg/kg). Blood samples were obtained by venipuncture at 1.5, 3, 4.5, 6, 7.5, 9, 10.5, 12, 13.5, 15, 16.5, 18 and 24 hours post-dosing and analyzed for acyclvoir concentration by HPLC. The animals were X-rayed at each time point to track the transit of macrospheres. The maximum serum concentration (Cmax) for Formulation 1 was 20.5±3.6 μg/ml (mean±SEM, n=14) and the Cmax for Formulation 2 was 26.7±7.1 μg/ml (mean±SEM, n=12). The maximum serum concentration was reached between 3-4.5 hrs post-dosing (Tmax) for both formulations. Therapeutic serum concentrations were maintained for a minimum of 15 hrs post-dosing.

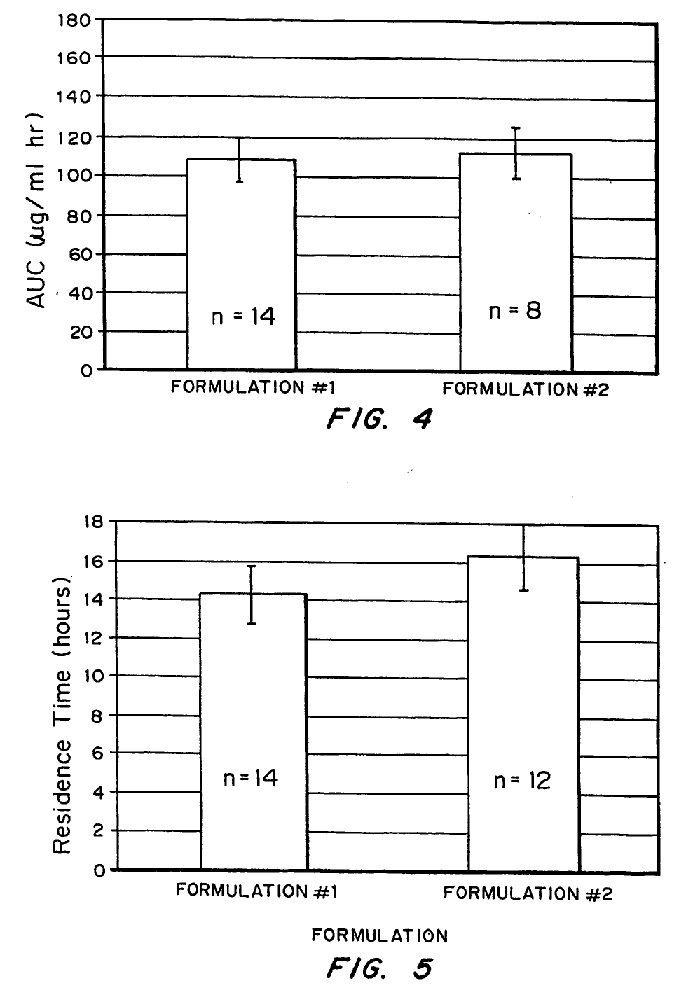

The “area under the serum concentration versus time curves” (AUC) displayed in FIG. 4 were calculated using Prism software. Formulation 1 had an AUC of 107±11 μg/ml*hr (mean±SEM, n=14) and Formulation 2 had a similar AUC of 111±13 μg/ml*hr (mean±SEM, n=12).

The residence time of macrospheres in the “upper GI” of dogs (stomach and small intestine) was determined by analysis of x-rays. The results are shown in FIG. 5. Formulation.1 had an upper GI residence time of 14.2±1.5 hr (mean±SEM, n=14) and Formulation 2 had a similar residence time of 16.2±1.8 hrs (mean±SEM, n=12).

### EXAMPLE 7

### Production of Multi-Drug Macrospheres

Fluidized bed spraying of 5% RL 100-coated 40% Acyclovir (ACV) loaded cores with 25% sodium salicylate w/w in a 10% RL 100-coating was then used to produce multi-drug macrospheres.

The starting material was the product of Example 2 (5% w/w RL 100 coated 40% ACV cores and overcoat with 10% RL 100 coating containing 25% w/w sodium salicylate). Overcoating with a 10% w/w coating of RL100 containing 25% w/w salicylate was used to produce a biphasic drug system. Sodium salicylate should be quickly delivered followed by acyclovir release. A 176.0 g lot of beads, 40% w/w Acyclovir (1.4-2.36 mm) was used as the cores for the coatings.

TABLE 4
COMPOSITION OF EUDRAGIT ® RL 100 COATING

| | |
|---|---|
| Liquid | Solid |

| Component | gm | w/w | gm | w/w |
|---|---|---|---|---|
| Eudragit ® RL 100 | 5.9 | 3.25% | 5.9 | 16.91% |
| DBS | 0 | 0.00% | 0 | 0.00% |
| Talc | 20 | 11.03% | 20 | 57.31% |
| Mg Stearate | 0 | 0.00% | 0 | 0.00% |
| DCM | 146.5 | 80.76% | | 0.00% |
| IPA | 0 | 0.00% | | 0.00% |
| Sodium Salicylate | 9 | 4.96% | 9 | 25.79% |
| Total | 181.4 | 100.00% | 34.9 | 100.00% |

The beads were fluidized at 200 fps with an inlet air temperature of 89° F. using the Wurster setup. The 10″ Wurster tube was used; and set 1″ from the top of the spray nozzle. The coatings were sprayed at an atomization pressure of 10 psi. The formulation exhibited a weight gain of 17.4 g (9.9%). The beads were dried in the fluidized bed for 5 min. The coatings appeared thin and uniform.

Multiple attempts were made to spray this formulation, all of which failed. The beads coalesced after a few minutes of spraying and could not be fluidized. It was determined that sodium salicylate was partially soluble in IPA and acted as a plasticizer. To counteract this phenomenon, DBS and IPA were omitted and the amount of talc was increased by 4 fold. The resulting improved formulation sprayed perfectly.

### EXAMPLE 8

### Release Kinetics from Multi-Drug Macrospheres

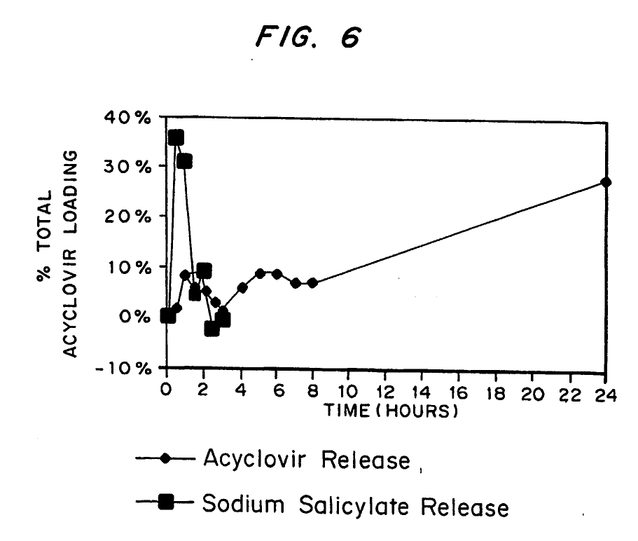

The release kinetics of the two drugs from the macrospheres of Example 6 were then determined. FIG. 6 is a graph of release of acyclovir (ACV) and salicylate as a function of percent total acyclovir loading, over time (in hours), from macrospheres wherein the salicylate is encapsulated in an outer Eudragit® RL 100 coating and the acyclovir is encapsulated in the core. The outer drug loading is used to achieve rapid release (three hours) as compared to more long term release of the core drug (24 hours).

### EXAMPLE 9

### Scale Up Production of Acyclovir Cores

This example demonstrates the production of 40% drug-loaded sphere cores of MCC/HPC/BaSO₄ and lactose with a diameter size distribution between 1.4 mm and 2.36 mm, and establishes that a procedure which can be increased in scale.

Materials/Controls: Fresh extrusion mix was prepared. The dry solids listed below in Table 5 were combined in the Hobart mixer and mixed for 5 min at speed setting #1. Water was poured in and the mixture was stirred for 10 minutes on the low gear. The resulting mixture was free flowing and grainy. The granulation was stored in a sealed plastic bag at 4° C. overnight (16 hrs) and extruded in the morning.

TABLE 5
COMPOSITION OF 40% ACYLCLOVIR CORES

| | | | | | | w/w |
|---|---|---|---|---|---|---|
| | | | | Weight | w/w | Total |
| Material | Manufacturer | Catalog # | Lot # | (g) | Solids | Mix |
| Microcrystalline | Spectrum | CE112 | PX0066 | 351.9 | 35.0% | 25.6% |
| Cellulose (MCC) | | | | | | |
| Lactose | Spectrum | LA103 | PO0171 | 0 | 0.0% | 0.0% |
| Barium Sulfate | Fluka | 11845 | 409062/1 23600 | 231.3 | 23.0% | 16.8% |
| (BaSO₄) | | | | | | |
| Hydroxypropyl | Hercules | KLUCEL EF | 8622 | 14.3 | 1.4% | 1.0% |
| cellulose (HPC) | | | | | | |
| Acyclovir | Interchem | 1.41E+09 | Certificate 24965 | 400.2 | 39.8% | 29.1% |
| SLS | Spectrum | S133 | PP0623 | 8.3 | 0.8% | 0.6% |
| Water | | | | 370.3 | | 26.9% |
| Solids total | | | | 1006.0 | | 73.1% |
| Total | | | | 1376.3 | | 100.0% |

The bulk mixture was extruded on a Caleva Model 25 extruder with a 2 mm screen at 7 rpm. The bulk mixture appeared to be nearly optimal. The bulk mixture was spheronized in 2 batches on a Caleva Model 250 spheronizer using the coarse plate (pitch size 4.5 mm) at 1000 rpm for 10 minutes. The spheronized extrudate was separated based on size. The fines content (<0.5 mm) was 1.8 μg (0.2%). The spheronized extrudate was tray-dried in a conventional oven at 50° C. overnight. The dry spheres were separated based on size (mm), weight (gm), and yield (%w/w): (a) <0.5, 1.8, 0.18%; (b) 0.5-1.4, 150.5, 14.96%; (c) 1.4-2.36, 769.6, 76.51%; and (d) >2.36, 27.2, 2.70%. The total recovery from raw materials was 949.1 g (94.4%).

1. A bioadhesive macrosphere for administration to the gastrointestinal tract or other mucosal lined lumen comprising
a core comprising a therapeutic, diagnostic or prophylactic agent,
agent release rate controlling means, and
a bioadhesive coating effective to increase retention to the mucosal lining of the gastrointestinal tract or mucosal lined lumen.

2. The macrosphere of claim 1 having dimensions between 0.1 and 3 mm in diameter.

3. The macrosphere of claim 1 wherein the agent is not well absorbed in the colon when orally administered.

4. The macrosphere of claim 3 wherein the agent is acyclovir.

5. The macrosphere of claim 1 comprising multiple agents.

6. The macrosphere of claim 1 comprising multiple bioadhesive or release rate controlling coatings.

7. The macrosphere of claim 1 comprising between 10 and 70% of a therapeutic, diagnostic or prophylactic agent by weight of macrosphere, or between 30 and 90% by weight of the core of the macrosphere, wherein each coating makes up between 1 -10% by weight of the macrosphere, up to a total of about 30% by weight of the macrosphere.

8. The macrosphere of claim 1 wherein the coating comprises agent in a ratio of between 5 and 50% by weight of the coating, preferably between 20 and 40% by weight of the coating, while still retaining rate control.

9. The macrosphere of claim 1 wherein the bioadhesive is selected from the group consisting of oligomers, metal oxides, peptide or protein ligands, saccaride ligands, and bioadhesive polymers.

10. The macrosphere of claim 1 in a tablet.

11. The macrosphere of claim 1 in a capsule or enteric coating.

12. A macrosphere comprising a core comprising a prophylactic, therapeutic or diagnostic agent and an outer bioadhesive or release rate controlling coating, having agent incorporated into and released from the bioadhesive or release rate controlling coating.

13. A bioadhesive system for release of a therapeutic, diagnostic or prophylactic agent in the gastrointestinal tract or other mucosally lined lumen comprising
a bioadhesive macrosphere or tablet comprising
a core comprising a therapeutic, diagnostic or prophylactic agent,
agent release rate controlling means, and
a bioadhesive coating effective to increase retention to the mucosal lining of the gastrointestinal tract or mucosal lined lumen, wherein the coating comprises agents for rapid release of the agent.

14. The system of claim 13 wherein the system comprises gas-generating means activated by exposure to water.

15. The system of claim 14 further comprising a coating preventing release until the system reaches the stomach or small intestine.

16. (canceled)

17. A method of delivering a therapeutic, diagnostic, or prophylactic agent comprising administering to a patient in need thereof a composition comprising a bioadhesive macrosphere and a pharmaceutically acceptable carrier, wherein the macrosphere comprises
a core comprising a therapeutic, diagnostic or prophylactic agent,
agent release rate controlling means, and
a bioadhesive coating effective to increase retention to the mucosal lining of the gastrointestinal tract or mucosal lined lumen.

18. The method of claim 17, wherein the macrosphere is administered via the nose, mouth, rectum, or vagina.

19. The method of claim 17, wherein the macrosphere comprises multiple agents.

20. The method of claim 17, wherein the macrosphere comprises multiple bioadhesive or release rate controlling coatings.
